(19) United States
(12) Patent Application Publication
(10) Pub. No.: US 2026/0335503 A1
(43) Pub. Date: Oct. 29, 2026

(54) BIOMATERIAL-INK FOR 3D PRINTING

(71) Applicant: UNIVERSITY OF CENTRAL FLORIDA RESEARCH FOUNDATION, INC., Orlando, FL (US)

(72) Inventors: Bradley Jay WILLENBERG, Saint Cloud, FL (US); Thomas KEAN, Orlando, FL (US); Andrew PANARELLO, Orlando, FL (US)

(21) Appl. No.: 19/111,559

(22) PCT Filed: Sep. 27, 2023
(86) PCT No.: PCT/US2023/033827

§ 371 Date: Mar. 13, 2025

Related U.S. Application Data
(60) Provisional application No. 63/410,352, filed on Sep. 27, 2022.

(51) Int. Cl. A61L 27/20; A61L 27/22; A61L 27/38; B33Y 10/00; B33Y 70/00

(52) U.S. Cl. CPC A61L 27/20; A61L 27/222; A61L 27/38; B33Y 10/00; B33Y 70/00

(57) ABSTRACT

Three-dimensional (3D) bioprinting has tremendous potential to aid in the creation of engineered tissues and organs that are so desperately in need. In this disclosure, a new type of bio-ink for 3D bioprinting made of sheared slurries of biomaterial hydrogel, in particular alginate hydrogel having micro-capillary structure (Capgel), in particular Capgel having increased stackability and thus self-assembly capability through the formation of a polyelectrolyte complex “skin” on the outer surfaces of the Capgel block with poly-L-lysine (PLL) coating prior to printing needle extrusion is provided as injectable biomaterial tissue scaffolding system, so that such stackable structure as weave patterns and cylindrical structures can be successfully printed with the new Capgel bio-ink. Further, the micro-capillary structure inherent in the original Capgel blocks is retained in the printed bio-ink making up the larger 3D structures and allows for cell culture in it.

### BACKGROUND

### Field of the Invention

The disclosed invention relates to a material to be used as biomaterial ink for 3D bio-printing, in particular to bio-ink comprising the sheared slurries of alginate gels having micro-capillary structure (Capgel) and/or self-healing capabilities.

### BACKGROUND OF THE INVENTION

There is a growing need for functional replacement of tissues and organs. The advent of tissue engineering and use of tissue scaffolds to create functional transplants in vitro is an innovative breakthrough for the medical community with great potential to address the shortfalls in donor/tissue organ supplies.

Three-dimensional (3D) printing and bioprinting have critically expanded tissue engineering approaches and made it possible to produce constructs that better replicate the complex structure and function of natural tissues and organs. Such 3D printed/bio-printed tissues and organs could also potentially reduce the use of animal models in research by substitution with engineered tissues that closely mimic physiologies of interest with human rather than animal cells.

However, there are challenges however encountered with 3D printing in tissue engineering often due to the paucity of biomaterial bio-inks that serve as the initial extracellular matrix (ECM) of printed tissue/organs, limited resolution of printers, and poor vascularization of the resulting 3D-constructs. Hence, there is a need for new 3D printing biomaterial bio-inks that are readily extruded (i.e., printable), self-supporting and adhesive. 3D printing bio-inks should possess appropriate, tunable bioactivity and porosity to facilitate robust colonization by target cells (i.e., biocompatible).

### SUMMARY OF THE INVENTION

The embodiments of the invention present a biomaterial-ink for 3D printing and 3D bioprinting applications, in particular Capgel biomaterial-ink comprising sheared slurries of alginate gels having pre-formed micro-capillary structures that are retained and thereby incorporated into the 3D printed/bioprinted structure. According to specific aspects, provided is a poly-L-lysine coated Capgel with enhanced stackability and thus self-assembly.

Various Capgels have been successful in wide range of tissue engineering applications including 3D stem cell culture scaffolds [Willenberg, B. J. Z. et al., J Biomed Mater Res A, 2006, 79(2):440-50], injectable stem cell delivery [Willenberg, B. J. Z. et al, J. Biomater. Sci. Polym. Ed., 2011, 22(12):1621-1637], in vitro construction of functional nerve [Anderson, W. A. et al., J Neurosci Methods, 2018, 305:46-53; George, D. S. et al., J Tissue Eng Regen Med, 2019, 13(3):385-395], and as injectable wound healing biomaterials [Bosak, A. et al., International Journal of Polymeric Materials and Polymeric Biomaterials, 2018. 68(18):1108-1117; Rocca, D. G. e al., Int J Cardiol, 2016, 220:149-54].

Capgels are a unique family of self-assembled hydrogel biomaterials characterized in that the gel comprises a plurality of continuous parallel microtubular capillaries having a first end and a second end, that the diameter of each of the continuous parallel microtubular capillaries is within the range of about 10 μm to about 300 μm, that the diameters of each of the continuous parallel microtubular capillaries may be different, that any one diameter may vary between the first end and the second end, and that the cross-section of each of the continuous parallel microtubular capillaries is non-circular.

The primary component of the hydrogels disclosed herein is alginate, a natural anionic linear polysaccharide biopolymer composed of β-D-mannuronic and α-L-guluronic acids residues. Initial ionic crosslinking of an alginate solution via uniaxial diffusion of divalent metal ions such as Cu²⁺ generates the Capgel self-assembled micro-capillary structure. Capillary diameter and density can be tailored for a given application via selection of the initial alginate and/or diffusing divalent metal ion (i.e., Cu²⁺) concentrations [Axpe, E. and M. L. Oyen, Int J Mol Sci, 2016, 17(12): 1976; Lee, K. Y. and D. J. Mooney, Prog Polym Sci, 2012, 37(1):106-126]. Since alginate does not have cell attachment sites, gelatin, which has intrinsic Arg-Gly-Asp (RGD) cell-adhesion motifs, is added to the initial alginate solution [Gungor-Ozkerim, P. S. et al, Biomater Sci, 2018, 6(5):915-946; Neufurth, M. et al., Biomaterials, 2014, 35(31):8810-8819; Zhang, T., K. C. Yan, L. Biofabrication, 2013, 5(4):045010]. After ionic crosslinking, these gels are sectioned into blocks, subjected to carbodiimide chemistry to form peptide crosslinks to produce Capgel scaffolds. These scaffolds are then cut into smaller Capgel pieces, and loaded into a syringe for printing/extrusion.

According to one embodiment, provided is a capillary alginate gel (Capgel) biomaterial-ink for 3D printing, comprised of alginate and, optionally, a biopolymer, wherein the biopolymer is, optionally, gelatin. The Capgel comprises one or more of the following features:
the Capgel is in a 3-D form comprising at least one surface that is coated with a polyelectrolyte(s) and/or polyelectrolyte complex(es), wherein the polyelectrolyte is optionally, poly-L-lysine comprising a medium molecular weight (MW=30-70 kDa) positively-charged poly-L-lysine (about 0.01% to about 0.1%, w/v, or, optionally, about 0.05% w/v),
the Capgel comprises a plurality of continuous parallel microtubular capillaries having a first end and a second end,
the diameter of each of the continuous parallel microtubular capillaries is within the range of about 10 μm to about 300 μm,
the diameters of each of the continuous parallel microtubular capillaries may be different,
any one diameter of each of the continuous parallel microtubular capillaries varies between the first end and the second end, and/or
the cross-section of each of the continuous parallel microtubular capillaries is non-circular.

In a further embodiment, the Capgel further comprises a plurality of cells.

According to another embodiment, disclosed is a method of making a Capgel biomaterial-ink for 3D printing that includes the following steps:
a. adding degraded alginate to a biopolymer solution to form an alginate/biopolymer solution;
b. disposing the alginate/biopolymer solution in a container;
c. applying a copper containing solution to the alginate/biopolymer solution; and
d. subjecting the alginate/biopolymer solution from step (c) under conditions to permit the alginate/biopolymer solution to set into a capillary alginate gel (Capgel).

In a specific embodiment, the alginate/biopolymer solution comprises a wt % ratio of 0.5-1.0:1.0-2 alginate to biopolymer. In a more specific embodiment, the biopolymer is gelatin. In a related embodiment, the alginate/biopolymer solution comprises about 2% alginate and about 2.6% gelatin. The container in which the alginate/biopolymer solution is disposed may be coated with dehydrated alginate. In another example, the source of copper in the copper containing solution is copper sulfate. Accordingly, the method may further involve covering the container with a porous cover layer soaked with copper (II) sulfate solution. Upon forming the Capgel, the Capgel may be cut into segments and cross-linked using carbodiimide chemistry.

In another specific method embodiment, disclosed is a method of making a Capgel biomaterial-ink for 3D printing, the method comprising steps of:
a. degrading a gelatin solution;
b. adding alginate to the gelatin solution to form an alginate/gelatin solution comprising about 2% alginate and about 2.6 % gelatin;
c. disposing the alginate/gelatin solution in a container coated with dehydrated alginate;
d. covering the container with a porous cover layer soaked with copper (II) sulfate solution;
e. applying copper sulfate solution to the alginate/gelatin solution through the porous cover layer; and
f. allowing the alginate/gelatin solution to set into a capillary alginate gel (Capgel).

The formed Capgel may be cut into segments; and the Capgel segments are cross-linked via carbodiimide chemistry. In another related specific example, the Capgel is coated with a polyelectrolyte complex such as, but not limited to, poly-L-lysine. In a more specific example, the method involves a. sectioning the Capgel into about 2× about 2× about 2 mm pieces, and putting the pieces in a tube; b. adding sterile diluted solutions of medium molecular weight (MW=30-70 kDa) positively-charged poly-L-lysine (about 0.01% to about 0.1%, w/v, or, optionally, about 0.05% w/v) to the tube, and c. rotating the tube on an orbital shaker, overnight, at room temperature.

The preceding methods may further comprise loading segments of Capgel into an extrusion device comprising a reservoir and a needle. Capgel may be extruded using the loaded extrusion device, typically onto a substrate, which forms stabilized sheared slurries typically possessing capillary structures of random alignment.

According to another embodiment, disclosed is a use of a Capgel biomaterial-ink for 3D printing made according to the disclosed embodiments for producing poly-L-lysine coated Capgel particles as microspheres and microcapsules for encapsulation and delivery of drugs and cells or as injectable biomaterial tissue scaffolds. According to a specific embodiment, the method further involves loading a plurality of cells into the particles of sheared slurries of Capgel biomaterial-ink prior to extruding. Alternatively, the method involves applying cells to the extruded Capgel.

As disclosed herein, it has been demonstrated that the self-assembled micro-capillary structure inherent in the original Capgel blocks was retained in the printed gel slurries of individual microgel particles (FIG. 1).

In other embodiments, to characterize the relationship between the inner diameters of various needles used to extrude Capgel and the resultant bulk and microscopic properties of the extruded biomaterial-ink slurries, extrusions of Capgel through needles (0.1-0.8 mm inner diameter) were investigated. It was established that extruded biomaterial-ink line widths and particle sizes were both functions of needle inner diameter (FIG. 2 and FIG. 3).

In other embodiments, the printability, stackability and self-supporting capacities of Capgel biomaterial biomaterial-ink were assessed by printing various 3D print structures (FIG. 4).

Further, in order to enhance stackability and thus self-adherent capability of the Capgel biomaterial-ink, it was discovered that after coating the Capgel blocks with poly-L-lysine (PLL) and extruding the PLL-coated Capgel blocks through a needle, the fragmented and sheared particles fracture and thereby exposing cryptic sites of negatively-charged carboxylic groups of internal alginate. Further, it was observed that these negatively-charged groups are capable of forming new polyelectrolyte bonds with areas of the positively-charged poly-L-lysine skin on neighboring entangled particles, thereby forming stabilized Capgel extruded slurries.

According to yet another embodiment, disclosed is a method of 3-D printing, comprising loading a Capgel biomaterial-ink into an extrusion device comprising a reservoir and, optionally, syringe barrel with tip/needle, and extruding the Capgel biomaterial-ink onto a surface, wherein the Capgel biomaterial-ink is comprised of alginate and optionally a biopolymer, wherein the biopolymer is, optionally, gelatin. In specific embodiments, the Capgel optionally (i) is in a 3-D form comprising at least one surface that is coated with a polyelectrolyte(s) and/or polyelectrolyte complex(es), the polyelectrolyte optionally comprising poly-L-lysine, (ii) includes a plurality of continuous parallel microtubular capillaries having a first end and a second end, (iii) the diameter of each of the continuous parallel microtubular capillaries is within the range of about 10 μm to about 300 μm, (iv) the diameters of each of the continuous parallel microtubular capillaries may be different, any one diameter of each of the continuous parallel microtubular capillaries varies between the first end and the second end, and/or (v) the cross-section of each of the continuous parallel microtubular capillaries is non-circular.

Another embodiment pertains to capillary alginate gel (Capgel) biomaterial-ink for 3D printing, comprised of alginate and optionally a biopolymer(s), wherein the biopolymer is, optionally, gelatin, and wherein the Capgel is in a 3-D form comprising at least one surface that is coated with a polyelectrolyte(s) and/or polyelectrolyte complex(es), wherein the polyelectrolyte is optionally, poly-L-lysine comprising a medium molecular weight (MW=30-70 kDa) positively-charged poly-L-lysine (about 0.01% to about 0.1%, w/v, or, optionally, about 0.05% w/v). In more specific embodiments, the Capgel
comprises a plurality of continuous parallel microtubular capillaries having a first end and a second end,
the diameter of each of the continuous parallel microtubular capillaries is within the range of about 10 μm to about 300 μm,
the diameters of each of the continuous parallel microtubular capillaries may be different,
any one diameter of each of the continuous parallel microtubular capillaries varies between the first end and the second end, and/or
the cross-section of each of the continuous parallel microtubular capillaries is non-circular.

In other embodiments, the Capgel was coated with poly-L-lysine prior to extrusion (FIG. 5 and FIG. 6), i.e., shear-induced fragmentation poly-electrolyte bonding (SIFPeB). This novel approach resulted in continuous, self-adherent extrusions that remained intact in solution.

In addition, in other embodiments, the biocompatibility of this new Capgel-PLL biomaterial-ink was evaluated for the ability to sustain a culture of human lung fibroblasts (HLFs), a cell type known to support and facilitate vascular formation and stability in co-culture with endothelial cells. This work represents the start of an innovative new avenue for future 3D-printing and 3D-bioprinting investigations.

### BRIEF DESCRIPTION OF THE DRAWINGS

FIG. 1. Capillary alginate hydrogel (Capgel) biomaterial tissue scaffolds have uniform microstructures of parallel, patent, regular tubular microchannels. (A) Gross stereomicrograph of cut Capgel pieces used to load syringe barrels. Phase-contrast micrographs of Capgel (B) imaged parallel to and (C) perpendicular to the micro-capillary structure. (D) Average capillary diameter and density of Capgel used in this study±standard deviation (SD). Scale bar=1 mm for (A) and 100 μm for (B & C)

FIG. 2. Widths of Capgel biomaterial biomaterial-ink extrusions from a range of different gauge needles follow a power-law relationship. (A-F) Stereomicrographs of Capgel needle extrusions; needle gauge used for each is indicated in the upper right of each panel. (G) Plot of average extruded line widths as a function of needle inner diameters (i.e., gauges). Scale bar=5 mm for (A-F) and error bars=SD in (G).

FIG. 3. Sizes (areas) of sheared Capgel microparticles from extrusions using a range of different gauge needles follow a power-law relationship. (A-F) Phase-contrast micrographs of sheared Capgel microparticles formed during needle extrusions; needle gauge used for each is indicated in the upper right of each panel. Insets show representative stereomicrographs of multiple sheared Capgel microparticles resulting from extrusion through each corresponding needle gauge. (G) Plot of average microparticle areas (i.e., sizes) of extruded Capgel microparticles as a function of needle inner diameters (i. e, gauges). Scale bar =100 μm for (A-F) and 1 mm for all insets; error bars=SD in (G)

FIG. 4. Capgel biomaterial biomaterial-ink can be used to print 3D structures of varying complexity. Images of (A) a 10×10 mm weave/mesh pattern, (B) a 10 mm-diameter cylindrical structure, (C) a single feather of the Pegasus graphic, and (D) a 10 mm-diameter Pegasus graphic. Scale bar=2.5 mm for all.

FIG. 5. Capgel-PLL biomaterial biomaterial-ink self-adheres through shear-induced fragmentation followed by polyelectrolyte bonding. Capgel 25 G needle extrusions before (A) and after (B) submersion in saline. (C) Illustration of the Shear-Induced Fragmentation Polyelectrolyte Bonding-SIFPeB-mechanism for Capgel-PLL self-adherence. Capgel-PLL 25 G needle extrusions before (D) and after (E) submersion in saline. Scale bar=1 mm for all.

FIG. 6. Microgels that comprise Capgel and Capgel-PLL needle extrusions retain the micro-capillary structure present in each biomaterial prior to extrusion. Stereomicrograph 3D reconstructions of (A) Capgel and (B) Capgel-PLL cut pieces prior to extrusion. Representative areas of the micro-capillary structures of each hydrogel piece are outlined with white ellipses; solid-line ellipses highlight the capillary microarchitecture viewed parallel to the capillary long-axis and dashed-line ellipses highlight capillary microarchitecture viewed perpendicular to this axis. Stereomicrograph 3D re-constructions of (C) Capgel and (D) Capgel-PLL 25 G needle extrusions; the different ellipses highlight the same orientations described above and the white dashed lines highlight borders between entangled microgels.

FIG. 7. Human lung fibroblasts (HLFs) attach and spread on Capgel-PLL biomaterial biomaterial-ink extrusions in culture. (A) Large-area maximum z-projection confocal fluorescence mosaic micrograph composed of multiple contiguous image fields (10× mag) stitched together of HLFs colonizing a Capgel-PLL extrusion taken at one week in culture. (B) Same fluorescence micrograph as shown in (A) merged with the corresponding differential interference contrast (DIC) micrograph of the Capgel-PLL extrusion. (C) Maximum z-projection confocal fluorescence and (D) DIC micrographs of a microgel particle colonized by HLFs in another Capgel-PLL extrusion taken at one week in culture. (E) Polar plot of HLF nuclei orientations relative to the capillary long-axis from individual microgel particles in extruded Capgel-PLL like that shown in (C) and (D) colonized with cells that attached and spread during a one-week culture. Green fluorescence is from actin filaments stained with a conjugated phalloidin dye (Actingreen 488) and blue fluorescence is from HLF nuclei stained with NucBlue. Scale bar =200 μm for (A & B) and 100 μm for (C & D).

### DETAILED DESCRIPTION

### Definitions

The following definitions are provided to better define the present disclosure and to guide those of ordinary skill in the art in the practice of the present disclosure. Unless otherwise noted, terms are to be understood according to conventional usage by those of ordinary skill in the relevant art.

As used in this specification and the appended claims, the articles “a”, “an’, and “the” refer to one or to more than one (i.e., to at least one) of the grammatical object of the article, unless the context clearly dictates otherwise. Thus, for example, “a scaffold’ refers to more than one such scaffold, “a capillary’ refers to more than one such capillary, “a biological or biologically active agent’ refers to more than one such agent, “a cell’ refers to more than one such cell, and the like.

As used herein, “about” refers to a value that falls within a 10-30% variance of the stated value. For example, an about 50% alginate solution refers to 50% alginate or a range of 35-65% alginate solution.

As used herein, “Capgel” refers to capillary alginate gel. Alginate is extracted as sodium alginates from brown seaweed, and alginate gel is commonly used as a binding, stabilizing and/or thickening additive gel due to their biocompatibility, nontoxicity, biodegradability, low-cost, and being simple to produce, and particularly valued for its application in foods and cosmetics [ISP Alginates, Section 3. Algin-Manufacture and Structure, in Alginates. Products for Scientific Water Control, 2000, International Specialty Products: San Diego, pp. 4-7]. Clinically, alginate is used in dental impression materials and hemostatic wound dressings [Blair, S. D. et al., Brit. J. Surg., 1990, 77(5):568-570; Rives, J. M. et al., Wounds—a Compendium of Clinical Research and Practice, 1997, 9(6):199-205].

Alginate gel is a linear polysaccharide of polymer chain, i.e., a linear copolymer with homopolymeric blocks of (1→4)-linked β-D-mannuronate (M) and α-L-guluronate (G) residues, respectively, covalently linked together in different sequences or blocks. The monomers may appear in homopolymeric blocks of consecutive G-residues (G-blocks), consecutive M-residues (M-blocks) or alternating M and G-residues (MG-blocks). Compositional variation is a reflection of source and processing. The pKa's of the C5 epimers are 3.38 and 3.65 for M and G respectively with the pKa of an entire alginate molecule somewhere in between [Schuberth, R. Ionotropic Copper Alginates. Investigations into the formation of capillary gels and filtering properties of the primary membrane, 1992, University of Regensburg: Regensburg; ISP Alginates, Section 3.Algin Manufacture and Structure, in Alginates. Products for Scientific Water Control, 2000,International Specialty Products: San Diego, pp. 4-7]. Alginate forms colloidal gels (high water content gels, hydrogels) with divalent cations such as Cd²⁺>Ba²⁺>Cu²⁺>Ca²⁺>Ni²⁺>Co²⁺>Mn²⁺, and among them Ca²⁺ is the best characterized and most used to form gels [Ouwerx, C. et al., Polymer Gels and Networks, 1998, 6(5):393-408].

As used herein, the term “copper capillary’ or “copper capillaries’ refers to the continuous parallel capillaries formed in the copper capillary alginate gels (CCAG) by allowing solutions of Cu²⁺ to diffuse uniformly into viscous solutions of alginate. These capillaries exhibit curved inner surfaces useful for seeding and propagating cells. The cross-section of the capillaries may be circular or non-circular. [Schuberth, R., Ionotropic Copper Alginates. Investigations into the formation of capillary gels and filtering properties of the primary membrane. 1992: Thiele, H., Histolyse und Histogenese, Gewebe und ionotrope Gele, Prinzipeiner Stukturbildung. 1967]. However, in common tissue culture media, CCAG alone swells, loses mechanical properties, and eventually dissolve due to a loss of copper ions that are released into the surrounding fluid environment. Accordingly, embodiments disclosed herein address a need for a modified CCAG that provides a stable tissue scaffold in a cell culture environment or within a human or animal.

As used herein, the term “stabilizing agent” refers to a compound, ion, or moiety that reacts with the CCAG so that the resulting stabilized CCAG maintains its mechanical properties in a cell culture or within a human or animal and the stabilized CCAG is non-toxic to its surrounding environments.

As used herein, the term “carbodiimide chemistry” refers to carbodiimide crosslinker chemistry. Carbodiimide conjugation works by activating carboxyl groups for direct reaction with primary amines via amide bond formation. For example, EDC (1-ethyl-3-(-3-dimethylaminopropyl) carbodiimide), in conjunction with NHS (N-hydroxysuccinimide) allows 2-step coupling of two proteins without affecting the carboxyls of the second protein. First, EDC activates carboxyl groups and forms an amine reactive O-acylisourea intermediate that spontaneously reacts with primary amines to form an amide bond and an isourea by-product.

As used herein, “bio-print” or “bio-printing” refers to three dimensional (3D) deposition of materials known as bio-inks to create tissue-like structures that are later used in various medical and tissue engineering fields, which is a techniques to combine cells, growth factors, and/or biomaterials to fabricate biomedical parts, often with the aim of imitating natural tissue characteristics.

As used herein, a “biomaterial-ink” refers to a composition suitable for 3D-printing and/or 3D-bioprinting comprising a biopolymer and, optionally, a plurality of cells. In this disclosure, it particularly refers to 3-D forms of Capgel that may be loaded into an extruding device to be extruded into stabilized biomaterial scaffolds. The biomaterial-ink may be printed to form a deposit of material to which cells are added. Alternatively, cells are added to biopolymer before printing, wherein the cells are extruded with a biopolymer.

As used herein, the term “scaffolds” or “bio-scaffolds” are three-dimensional (3D) porous, fibrous or permeable biomaterials intended to permit transport of body liquids and gases, promote cell interaction, viability and extracellular matrix (ECM) deposition with minimum inflammation and toxicity while bio-degrading at a certain controlled rate. Biomaterials such as collagen, various proteoglycans, alginate-based substrates and chitosan have all been used in the production of scaffolds for tissue engineering. Unlike synthetic polymer-based scaffolds, natural polymers are biologically active and typically promote excellent cell adhesion and growth.

As used herein, the term “electrostatically charged molecule’ refers to any molecule possessing functional groups (e.g., carboxylic acid or amine residues) in a charged state (e.g., ionized or protonated).

As used herein, the term polyelectrolyte refers to a cationic polymer possessing a positive charge that interacts with negatively charged polymers such as alginate. Poly-L-lysine and chitosan are examples of materials that form a polyelectrolyte complex. Other examples include Poly (vinylbenzyl trialkyl ammonium), Poly (4-vinyl-N-alkyl-pyridimiun), Poly (acryloyl-oxyalkyl-trialkyl ammonium), Poly (acryamidoalkyl-trialkyl ammonium), and Poly (diallydimethyl-ammonium).

As used herein, the term “poly-L-lysine (PLL)” refers to L-lysine homo-polypeptide belonging to the group of cationic polymers: at pH 7, poly-lysine contains a positively charged hydrophilic amino group. The precursor amino acid lysine contains two amino groups, one at the α-carbon and one at the ε-carbon. Either can be the location of polymerization, resulting in α-poly-lysine or ε-poly-lysine. Poly-L-lysine is a synthetic polymer of α-poly-lysine, which has the L chirality at lysine's central carbon.

As used herein, the term “culture’, or grammatical variations thereof, is intended to denote the maintenance or cultivation of cells in vitro, including the culture of single cells. Cultures can be cell, tissue, or organ cultures, depending upon the extent of organization.

All patents, patent applications, provisional applications, and publications referred to or cited herein are incorporated by reference in their entirety to the extent they are not inconsistent with the explicit teachings of this specification. Following are examples that illustrate procedures for practicing the invention. These examples should not be construed as limiting. All percentages are by weight and all solvent mixture proportions are by Volume unless otherwise noted.

### Overview

The embodiments of the invention present a bio-ink for 3D printing and 3D bioprinting applications, in particular Capgel biomaterial-ink comprised of alginate gels having pre-formed micro-capillary structures that are retained and thereby incorporated into the 3D printed/bioprinted structure, in particular a 3D form of Capgel having at least one surface coated with poly-L-lysine to form a polyelectrolyte complex “skin” on the outer surfaces of gel blocks prior to extrusion which surprisingly result in increased self-adherence between microgel particles in the slurries.

Capgels are a unique family of self-assembled hydrogel biomaterials characterized in that the gel comprises a plurality of continuous parallel microtubular capillaries having a first end and a second end, that the diameter of each of the continuous parallel microtubular capillaries is within the range of about 10 μm to about 300 μm, that the diameters of each of the continuous parallel microtubular capillaries may be different, that any one diameter may vary between the first end and the second end, and that the cross-section of each of the continuous parallel microtubular capillaries is non-circular.

The primary component of these hydrogels is alginate, a popular natural anionic linear polysaccharide biopolymer composed of β-D-mannuronic and α-L-guluronic acids residues. Initial ionic crosslinking of an alginate solution via uniaxial diffusion of divalent metal ions such as Cu²⁺ generates the Capgel self-assembled micro-structure. Capillary diameter and density can be tailored for a given application via selection of the initial alginate and/or diffusing divalent metal ion (i.e., Cu²⁺) concentrations. Since alginate does not have cell attachment sites, gelatin, which has intrinsic Arg-Gly-Asp (RGD) cell-adhesion motifs, is added to the initial alginate solution.

Capgel hydrogels were synthesized as previously described [Willenberg, B. J. Z. et al., J. Biomater. Sci. Polym. Ed., 2011. 22(12):1621-1637]. Briefly, the steps to make Capgel to obtain the sheared slurries of 3D biomaterial-ink comprise
a. degrading a gelatin solution with sodium hydroxide (NaOH);
b. adding alginate to the gelatin solution to form an alginate/gelatin solution comprising about 2% alginate and about 2.6 % gelatin;
c. disposing the alginate/gelatin solution in a container coated with dehydrated alginate;
d. covering the container with a porous cover layer soaked with copper (II) sulfate solution;
e. applying copper sulfate solution to the alginate/gelatin solution through the porous cover layer; and
f. allowing the alginate/gelatin solution to set into a capillary alginate gel (Capgel).

In certain embodiments, after ionic crosslinking, these gels are sectioned into blocks, subjected to carbodiimide chemistry to form peptide crosslinks, undergo a series of washes and then are terminally sterilized via autoclave to produce the final Capgel scaffolds. These scaffolds are then cut into smaller Capgel pieces and loaded into a syringe for printing/extrusion. In the context of the present disclosure, it is appropriate to consider Capgel as an elastomeric biomaterial ink, as it is a peptide-bond crosslinked network of alginate polysaccharide chains and gelatin polypeptide chains swollen in its presumed theta solvent water.

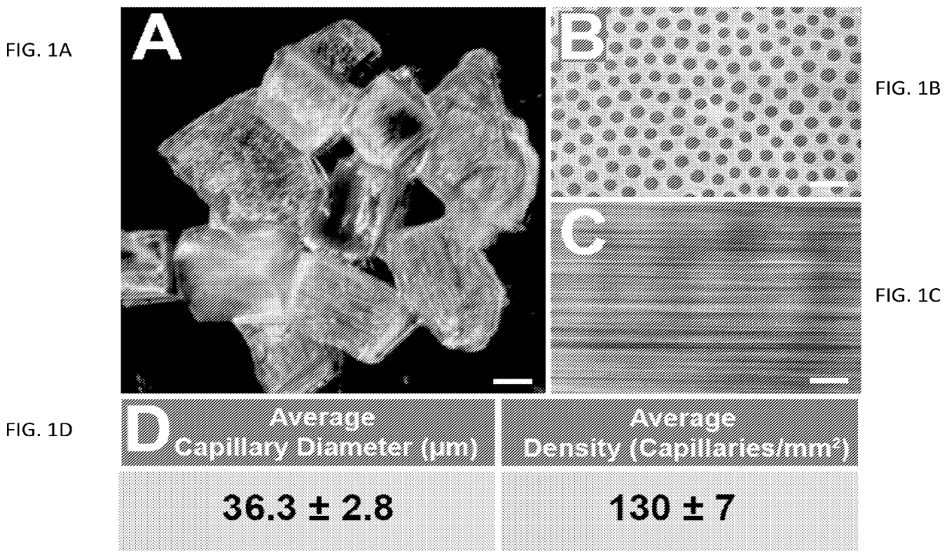

In some embodiments, it was shown that the self-assembled micro-capillary structure inherent in the original Capgel blocks was retained in in the printed gel slurries of individual complex and each entangled microgel particles (FIG. 1). The capillary microstructural characteristics of this Capgel are shown in FIGS. 1B & 1C; capillary diameters and density were 36.3±2.8 μm and 130±7 capillaries/mm² on average, respectively (FIGS. 1B & 1D).

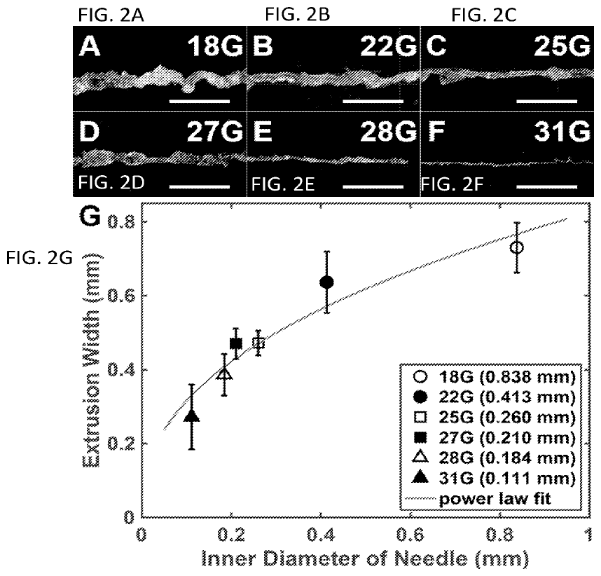

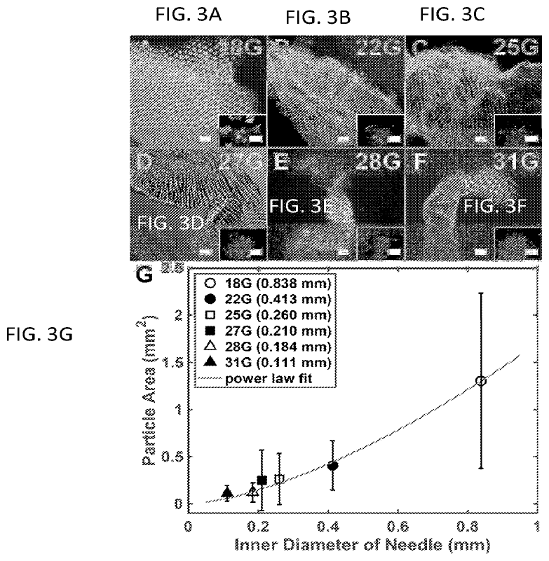

As described herein, the relationship between the inner diameters of various needles used to extrude Capgel and the resultant bulk and microscopic properties of the extruded biomaterial-ink slurries, extrusions of Capgel through needles (0.1-0.8 mm inner diameter) was investigated, and using different microscopy techniques combined with image analysis it was found that Capgel biomaterial-ink extrudes as slurries of fractured and entangled particles, each retaining micro-capillary structures, and that extruded biomaterial-ink line widths W and particle sizes A were both functions of needle inner diameter D, specifically power-law relationships of W~D^{0.42} and A~D^{1.52}, respectively (FIG. 2 and FIG. 3).

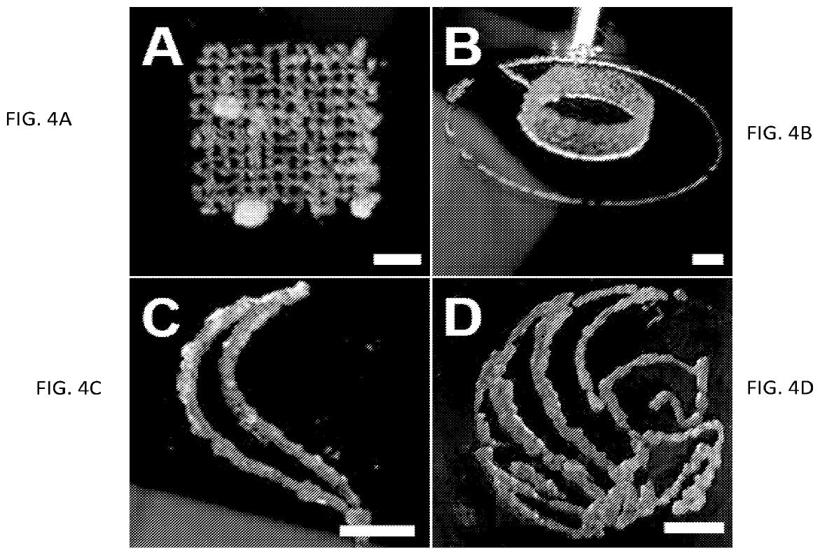

In other embodiments, the printability, stackability and self-supporting capacities of Capgel biomaterial biomaterial-ink were assessed by printing various 3D print structures (FIG. 4). It was demonstrated that the various constructs such as weave/mesh pattern, cylindrical structure and pegasus graphics that are 3D-printed with Capgel biomaterial-ink using 25 G- or 31 G needle syringe successfully supported their own weight.

Further, in order to enhance stackability and thus self-adherent capability of the Capgel biomaterial-ink, it was observed that after coating the Capgel blocks with poly-L-lysine and extruding poly-L-lysine-coated Capgel blocks through the needle, the fragmented and sheared particles fracture and thereby expose cryptic sites of negatively-charged carboxylic groups of internal alginate, which are capable of forming new polyelectrolyte bonds with areas of the positively-charged poly-L-lysine skin on neighboring entangled particles and that it forms a polyelectrolyte complex “skin” on the outer surfaces of the Capgel slurries.

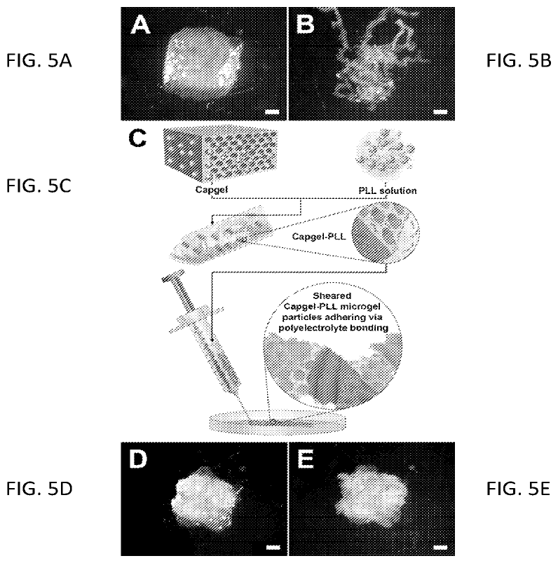

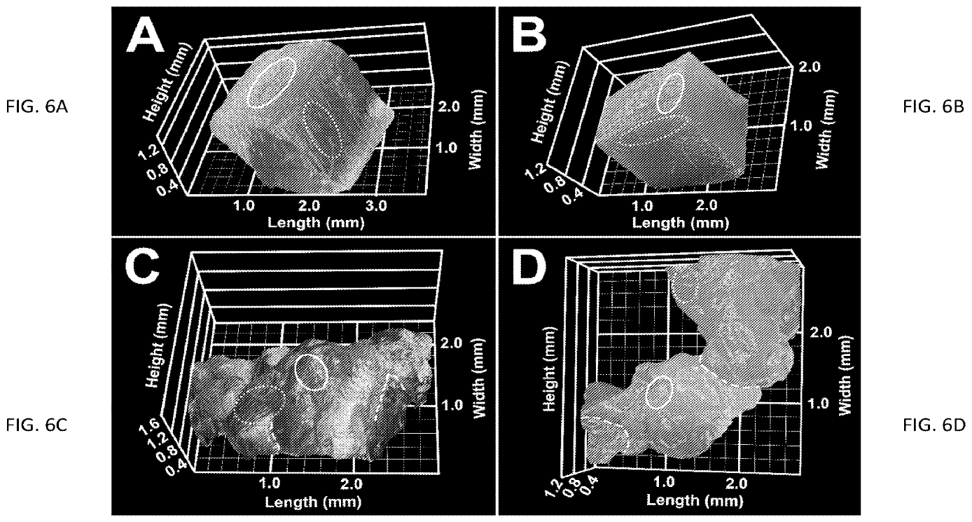

Typically, the Capgel is coated with poly-L-lysine prior to extrusion (FIG. 5 and FIG. 6), i.e., shear-induced fragmentation poly-electrolyte bonding (SIFPeB). Briefly, the steps are
a. sectioning the Capgel into about 2×2×2 mm pieces, and putting the pieces in a conical tube;
b. adding sterile diluted solutions of medium molecular weight (MW=30-70 kDa) positively-charged poly-L-lysine (about 0.05% w/v) to the conical tube, and
c. rotating the conical tube on an orbital shaker, overnight, at room temperature.

This novel approach resulted in continuous, self-adherent extrusions that remained intact in solution.

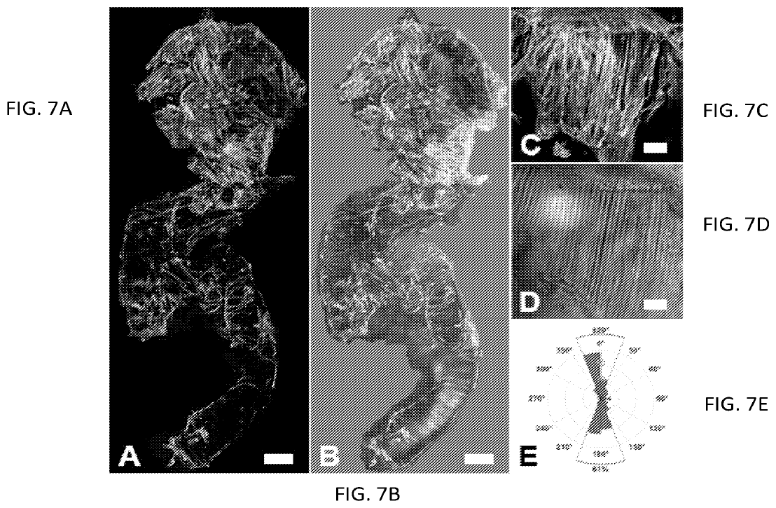

In addition, the biocompatibility of this new Capgel-PLL biomaterial-ink was then evaluated through the culture of human lung fibroblasts (HLFs), a cell type known to support and facilitate vascular formation and stability in co-culture with endothelial cells. In some embodiments, it was demonstrated that the produced random arrangement of microgels (biomaterial-ink slurries) could support the survival, attachment and spreading of cultured HLFs as well as induce cell orientation via the biomaterial-ink micro-capillary structure (FIG. 7). This work represents the start of an innovative new avenue for future 3D-printing and 3D-bioprinting investigations.

### EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention. Data and Figures presented herein are provided in Panarello et al, Gels 2022, 8(6):376.

### Example 1. Materials and Methods

### (1) Formation and Growth of Capgel Hydrogels

Bloom gelatin 10% (G1890, Sigma-Aldrich, MO, USA) was prepared in distilled, deionized water (ddH₂O) and degraded by heating with sodium hydroxide (NaOH) at 80 °C for 72 h. Oligomeric 10% gelatin solution was equilibrated to ambient temperature (~23 °C) for 2h. Sodium alginate powder (Protanal Pharm Grade LF10/60, FMC Biopolymer, PA, USA supplied by IMCD US) was then added to create a final solution of 2% alginate and 2.6% gelatin in ddH₂O.

Capgel parent gel was grown in a 10 cm glass petri dish coated with dehydrated 4% alginate. The parent solution was added to the alginate coated petri dish which was placed within a larger glass dish. The parent solution was covered with a 0.5M copper (II) sulfate (CuSO₄, Acros Organics, Flanders, Belgium) soaked Kimwipe which was held in place with a plastic ring and 0.5 M CuSO₄ was dripped over portions of the Kimwipe directly covering the growing parent gel. The Kimwipe was removed and the parent gel and petri dish were completely submerged in 0.5 M CuSO₄. After 72 h, the Capgel was cut into strips, which were then rinsed 3× in ddH₂O each day for four days in a covered plastic container. After ddH₂O washes, the strips were sectioned into ~5×5×3 mm blocks.

### (2) Crosslinking and Preparation of Capgel Blocks

Per 50 ml conical tube, four (4) ~5×5×3 mm Capgel blocks were crosslinked via carbodiimide chemistry in 20 mL PBS containing 1.89 mg/mL N-Hydroxysuccinimide (NHS) (ThermoFisher, MA, USA) which was allowed to soak into the gel overnight at 4 °C followed by adding 20 mL PBS with 1.57 mg/mL N-(3-Dimethyl-aminopropyl)-N′-ethylcarbodiimide hydrochloride EDC (Sigma-Aldrich, MO, USA) to yield a final reaction volume of 40 mL. The cross-linking reaction was then gently shaken overnight at 4 °C. After cross-linking, Capgel blocks were washed 3× with 0.2 μm filter-sterilized (564-0020, Nalgene, NY, USA) 0.9% NaCl (S271-3, ThermoFisher, MA, USA) in ddH₂O (sterile saline) over three days in a covered plastic container, followed by 3 washes with 0.2 μm filter-sterilized 10× sodium citrate solution (BP1325-4, ThermoFisher, MA, USA) over four days. Finally, the blocks were washed 3× in sterile saline over four days. Capgel in saline was then autoclaved with liquid cycle and sterilization hold of 15 min. Sterilized Capgel blocks were then stored at 4 °C in sealed glass bottles.

### (3) Capgel Capillary, Particle Size, and Extrusion Analysis

Average capillary size and density of used Capgel was found using NIH ImageJ software (v1.52b, National Institutes of Health, Bethesda, MD, USA). A sample size of 25 capillaries per gel was used to determine the average gel diameter of three separate samples. Using the circle tool in ImageJ, Capgel capillaries were outlined, and their diameters measured. These diameters were then averaged, and the standard deviation calculated.

Diced pieces of Capgel were loaded into the barrels of syringes with a range of different gauge needles using a sterile spatula (Corning, NY, USA) to determine the average size of the microgel particles produced by extruding the gel through the needle. The gauges (G) tested were 31 G, 28 G, 27 G, 25 G, 22 G, and 18 G. Syringe needles 18 G, 22 G, 25 G, and 27 G (Becton Dickinson, NJ, USA) were attached to a 1 mL Luer-Lok syringe (Becton Dickinson, NJ, USA). The 28 G and 31 G needles were fused to a 0.5mL fixed head tuberculin syringe (Becton Dickinson, NJ, USA). Sufficient amounts of Capgel were added to each syringe to fill ¼ to ½ of the total volume of the syringe. The gel was then extruded from each syringe onto a plastic petri dish (Corning, NY, USA) and were imaged using a stereo microscope and the images processed and analyzed using ImageJ. To determine extruded line widths, five points were selected in 3 mm intervals along the length of each extruded line and their thicknesses measured. For extruded microgel particle sizing, the method described by Ramalingam et al., was followed to capture the microgel particle area using ImageJ [Ramalingam, S. and V. Chandra, Marine Georesources & Geotechnology, 2017, 36(8):867-874]. Averages and standard deviations for extruded lines widths and microgel particle areas were calculated and power law relations were determined with MATLAB (vR2021b, Math Works, Natick, MA, USA).

### (4) 3D Printer Settings and Methods

In a biosafety cabinet, a Seed R3bel bioprinter was used to print the Capgel fragments through a blunt ended 25 G or 31 G needle. Stereolithography (STL) files were sliced (Slic3r v1.3.0, slic 3r. org), with settings of 3 mm filament diameter, an extrusion multiplier of 4X and a print speed of 30 mm/s, to create a gcode file for printer control. Pronterface software (Printrun v 1.6.0, pronterface.com) was used to control the desired print(s) on the bioprinter: an extrusion rate adjustment of 300%, and a print speed adjustment of 15% were then used. The structures were printed onto sterile 10 cm plastic petri dishes (Corning, NY, USA) and covered until it was time for imaging.

Four models were tested for printing with Capgel material. The first was a 1×1cm “weave” with a rectilinear infill pattern that was 2 layers high. The second model tested was a hollow cylinder with a 1 cm diameter, consisting of eighteen layers of height, which resulted in a height of ~3 mm. The third and fourth models came from the same pegasus source file. The third model was a “feather” from the Pegasus, while the fourth model was a 1 cm diameter print of the entire Pegasus model consisting of one extrusion layer.

### (5) Poly-L-Lysine Coating of Capgel (Capgel-PLL)

Capgel was coated with poly-L-lysine to create a polyelectrolyte complex (PEC) “skin” on the hydrogel (Capgel-PLL). Briefly, in a biosafety cabinet, sterilized Capgel blocks were transferred onto a sterile petri dish. A sterile scalpel was then used to dice the blocks into ~2×2×2 mm Capgel pieces. Sterile solutions of 0.05% (w/v), 30-70 kDa poly-L-lysine; Sigma-Aldrich, MO, USA) were made, and filter sterilized (Nalgene, NY, USA). Once sterilized, 10 mL of poly-L-lysine solution was added to a 15 ml conical tube together with diced Capgel pieces made from five (5) blocks and allowed to rotate on an orbital shaker (Orbitron Rotator II, Boekel, PA, USA), overnight, at room temperature. The Capgel pieces were then rinsed with three 30 min washes of 40 mL sterilized ddH₂O on orbital shaker. Depending on the experiment, the pieces were submerged in saline or DMEM cell culture media (Gibco, TX, USA) and allowed to soak at 37 °C overnight. Then Capgel-PLL pieces were loaded into 25 G tuberculin syringes (26046, EXELint, CA, USA) and extruded onto 10 cm plastic Petri dishes for imaging using a stereo microscope with digital camera (MU1803, AmScope, CA, USA), or extruded into culture plates for cell studies.

(6) Digital 3D Reconstruction of Capgel and Capgel-PLL pieces and extrusions

Optical micrographs and 3D reconstructions of Capgel and Capgel-PLL pieces and extrusions were acquired and generated using the Keyence VHX 7100 digital microscope with the Keyence VHX 7000 software (Keyence Corporation of America, Itasca, IL, USA). All images were acquired with E20 lens and ×80 digital zoom. The tilting angles of the lens with respect to the sample was 22° (Capgel piece), 23° (Capgel extrusion), −23° (Capgel-PLL piece) and −12° (Capgel-PLL extrusion). Samples were illuminated with a full ring light during image acquisition.

### (7) Cultivation and Preparation of HLF Cells

Normal human lung fibroblasts (NHLF CC-2512, Lonza, Basel, Switzerland) were cultured at 37 °C in T-75 culture flasks using Dulbecco's Modified Eagle Medium (DMEM) with 10% fetal bovine serum (FBS; Thermofisher, MA, USA), 1% Glutamax, and 1% penicillin/streptomycin (Gibco, TX, USA).

### (8) Post-Extrusion Cell Seeding of Extruded Capgel-PLL, Histological Staining, and Image Processing

Extruded Capgel-0.05% poly-L-lysine structures were seeded with normal HLFs (NHLF CC-2512, Lonza, Basel, Switzerland). This was accomplished by trypsinization (0.05%, Gibco, TX, USA) of the cells, centrifugation at 1912 RCF (Centra GP 8R, 216 4-place swinging bucket rotor, Thermofisher, MA, USA), and addition of 160,000 HLF cells over the entire extruded structure. The cells were then allowed to attach to the cell media-soaked print.

After 2 h, 2 mL of media was added to the Petri dish and the print was cultured at 37 °C for 1 week, changing media every 2 days. After 1 week, cellularized-Capgel was washed 3×in phosphate buffered saline (PBS; Sigma-Aldrich, MO, USA) before fixation with 4% par-aformaldehyde for 30 min at room temperature. Following fixation, cellularized-Capgel was rinsed 3× with PBS and then permeabilized with 0.2% Triton-X 100 for 30 min at room temperature. After 3 washes with PBS, cellularized-Capgel was stained with NucBlue Live and ActinGreen 488 ReadyProbes (Invitrogen; Carlsbad, CA) for 2 h in darkness. Finally, 3 rinses with PBS were performed prior to imaging on FluoroDish (FD35-100, World Precision Instruments Inc., FL, USA). Imaging was performed using the Zeiss 710 laser scanning confocal microscope at 10× magnification with excitation wavelengths of 405 nm and 488 nm for NucBlue Live and ActinGreen 488, respectively. Images were processed with Zen 2010 software (Zeiss; Jena, Germany).

Z-stacks of each channel (DIC, NucBlue, and actin-green) were overlaid and converted into maximal z-projections with ImageJ version 1.52b (National Institutes of Health; Bethesda, MD). Images were stitched together using Adobe Photoshop (2022 v23.3.2, Adobe Inc., CA, USA) and image background removed with magnetic lasso tool.

To determine orientation of HLF cell nuclei, measures were made of cell-colonized microgels that were, by chance, oriented with the capillary long-axis parallel to the imaging plane, which was readily determined by visual inspection of the corresponding DIC images. NucBlue channel images of these areas were rotated such that the long-axis of the capillaries were vertical. Images were then binarized by applying thresholding in ImageJ, and subsequently processed with the ‘region props’ function in MATLAB. Nuclei were approximated as elliptical blobs with major and minor axes. Nuclear orientation is that of the major axis with respect to the image vertical axis. Those nuclei with centroids above the horizontal midline of each image have possible orientation values of −90°-90°, with those below the midline having orientations 90°-270°.

### Example 2 (Capgel)

Capgel biomaterials formulated with 2% alginate/ 2.6% gelatin were successfully produced as previously described [Bosak, A. et al., International Journal of Polymeric Materials and Polymeric Biomaterials, 2018. 68(18): 1108-1117]. Capgel blocks cut into smaller pieces (FIG. 1A) were used as the biomaterial for the extrusion and printing experiments. Microstructural characteristics (FIGS. 1B & C) of this Capgel are shown in FIG. 1; capillary diameters and density were 36.3±2.8 μm and 130±7 capillaries/mm² on average, respectively (FIGS. 1B & D). In the context of the present studies, it is likely most appropriate to consider Capgel as an elastomeric biomaterial, as it is a peptide-bond crosslinked network of alginate polysaccharide chains and gelatin polypeptide chains swollen in its presumed theta solvent water.

### Example 3. Capgel Biomaterial-Ink Needle Extrusions

Capgel biomaterial-ink extrusion characteristics were determined over a wide range of needle gauges (FIG. 2). Analysis of optical micrographs of extruded lines of Capgel biomaterial-ink (FIG. 2A-F) shows that extruded line widths decrease with increasing needle gauge as expected (FIG. 2G). Further, it was determined that the linkage between Capgel extruded line widths (W) and needle inner diameters (D, i.e., gauge) is well-described by a power-law scaling relationship. Specifically, fitting all individual data points, W~D^{0.42} (R^{2=0.80}). The physics underlying the observed scaling are complex—Capgel is viscoelastic and is sheared and compressed through the extruder. Extruded line widths are also likely influenced by extrusion speed/volume and print X-Y translation speed. A more rigorous mathematical characterization of extrusion width with needle diameter that accounts for Capgel properties and extruder/extrusion dynamics is beyond the scope of the present study and will be a focus of future work. Notwithstanding, Capgel was demonstrated as a biomaterial-ink exhibits predictable extrusion line width across a range of needle diameters, and these widths have practical importance regarding gross dimensions and resolution of 3D prints with Capgel biomaterial-ink.

Microgels are defined as hydrogel microparticles. When Capgel blocks were forced through and extruded from fine gauge needles, lines of entangled microgels were formed due to compression and shearing (FIG. 2A-F). Submerging these resultant lines of extruded Capgel biomaterial-ink in saline dispersed these microgels, enabling imaging and quantification of the microparticle sizes (FIG. 3A-F; sizes described as the projected cross-sectional areas, mm2). These data show that average microgel size produced by the needle extrusions reduced dramatically over the 18 G to 31 G range (FIG. 3G). Fitting all data points, the projected microparticle area A (i.e., microparticle size) and needle inner diameter also follows a power-law scaling relationship, specifically A~D^{1.52} (R²=0.49, FIG. 3G). The value of the correlation coefficient is low because of the large variance in A produced by the largest needle; fitting only the average values of A yields R²=0.99. As shown in FIG. 3 as the inner diameter of the needle decreases, the variation of produced microparticle sizes also decreases (FIG. 3G), supporting the notion that higher gauge needles produce more consistent Capgel biomaterial-ink extrusions. The exceptionally large standard deviation values for the 18-gauge syringe may be due to the inherent elasticity of Capgel and the requisite strain and stress at facture to form the microgel fragments via shearing through a needle. This data also suggests that the 22 G needle inner diameter and the corresponding shear forces applied to Capgel during extrusion may represent threshold values required to produce extrusions with consistent (i.e., less variable) properties.

Extrusion characteristics and the coupling of these characteristics with needle gauge are also likely influenced by the size and shape of the Capgel pieces loaded into the syringe barrel for extrusion. It is also critically important to note that micro-capillary structure was retained in all microgel particles produced by all extrusion conditions (FIG. 3A-F), a vital property bolstering its use as a new 3D-printing biomaterial-ink. Capgel is viscoelastic and thus exhibits non-Newtonian and shear-thinning behavior. As such, the relation between shearing stress and rate of shearing strain is highly nonlinear. Furthermore, we posit as Capgel pieces are broken into smaller fragments by shear forces, the effective viscosity decreases. Thus, viscoelasticity is expected to be different for various needle gauges, prompting our investigation into the characteristics of Capgel extruded through 0.1-0.8mm diameter needles.

### Example 4. 3D Printing With Capgel Biomaterial-Ink

A variety of structures were successfully 3D-printed with Capgel biomaterial-ink including a weave/mesh pattern, an 18-layer cylindrical structure and pegasus graphic (FIG. 4). Based on the extrusion studies, all prints were executed with a 25 G tuberculin or 31 G insulin fused-needle syringe. The weave/mesh print was first attempted (FIG. 4A). The success of this print demonstrates that Capgel biomaterial-ink can print single-extrusion width lines in close proximity with minimal bleed and discontinuities. Next, a layered cylindrical structure was attempted to assess the stackability and self-supporting capacities of Capgel biomaterial-ink (FIG. 4B, Video S1). The success of this 3D print demonstrates that the biomaterial-ink as printed has sufficient self-adhesion/cohesion to stack in successive layers as well as support the weight of those layers. To investigate the potential of Capgel biomaterial-ink in a more elaborate 3D print with various curved and straight elements, a Pegasus graphic was produced. Once it was established that a single feather of the Pegasus could be printed (FIG. 4C, Video S2), the full graphic was attempted with success (FIG. 4D, Video S3).

It is evident that the greatest print fidelities were observed for the larger curved and circular elements present in the cylindrical and Pegasus prints. This potentially stems from tensile and compression forces at work when the 3D printer undertakes a dramatic change in direction, such as in the weave, and the interplay of those forces with the self-adhesivity/cohesivity and extrusion dynamics of the Capgel biomaterial-ink, which can result in print over-extrusions and/or discontinuities. These experiments helped to inform adjustments to printing parameters such as extrusion and X-Y translation speed to improve fidelity of future prints, although additional optimizations will need to be made to increase print fidelity for patterns with short runs and sharp angles such as elements in the weave/mesh and Pegasus prints. Overall, these prints demonstrate that 3D-prints containing lines of various curvatures, a wide range of angles and varying negative space elements can be achieved with Capgel biomaterial-ink.

### Example 5. Development of Capgel-PLL Biomaterial-Ink

The property of self-adherence is critical for biomaterial-inks to sustain the 3D-printed shape in solution. As shown in FIG. 5, Capgel extrusions created with a fine gauge needle resulted in a sheared slurries of entangled, fractured microparticles (FIG. 5A) that tended to disperse when submerged in an aqueous fluid volume (FIG. 5B).

The long-range order of the micro-capillary structure present in Capgel blocks (FIG. 6A) is disrupted in these extrusions, but this microarchitecture is preserved in each entangled microgel particle (FIG. 6C). To improve Capgel biomaterial-ink cohesion when submerged in aqueous fluids, a polyelectrolyte-complex “skin” was first created on Capgel prior to printing by soaking blocks in dilute (0.05% w/v), sterile solutions of medium molecular weight (MW=30-70 kDa) positively-charged poly-L-lysine.

The concept of self-adherent biomaterial-inks with Capgel-PLL is captured in FIG. 5C and was successful as shown by the improved cohesion evident for Capgel-PLL biomaterial-ink extrusions (FIGS. 5D & E). As with the Capgel extrusions, the long-range microstructural order of Capgel-PLL blocks (FIG. 6B) was interrupted by the extrusion process, but again, capillary microarchitectures were retained in each extruded microgel (FIG. 6D). Compared to Capgel biomaterial-ink, the Capgel-PLL formulation required greater force to extrude, which was beyond the capabilities of the basic 3D-printer utilized in this study. Future 3D-printing studies with Capgel-PLL biomaterial-ink are therefore planned using a printer with a wider range of capabilities.

### Example 6. Biocompatibility of Capgel-PLL Biomaterial-Ink

Evaluations of biocompatibility need to be undertaken for any new biomaterial formulation and/or application. Pursuant to this axiom, HLFs were seeded onto Capgel-PLL extrusions and cultured for a week. Following culture, cell-laden Capgel-PLL extrusions were fixed, treated with fluorescent dyes that stain actin filaments (phalloidin) and DNA (i.e., nuclei, DAPI) and imaged with laser-scanning confocal microscopy. Fluorescence and differential image contrast (DIC) images of these Capgel-PLL extrusions show that abundant HLFs had attached, spread and colonized the Capgel-PLL biomaterial-ink (FIG. 7A-D). As noted above, the microgel particles produced during extrusion preserve capillary orientation within the microparticles themselves, but each were randomly oriented upon exit from the needle (FIGS. 7B & D). Further, the cultured HLFs within the biomaterial-ink were strongly oriented by the Capgel-PLL micro-capillary structure (FIGS. 7C & D). Image analysis quantifying the nuclear orientation of cells colonizing these regions of the biomaterial-ink showed that 61% of the 397 nuclei imaged were aligned within +20° with respect to the Capgel-PLL capillary long axis (FIG. 6F). Taken together, these data strongly support the in vitro biocompatibility of Capgel-PLL biomaterial-ink and its tissue scaffolding potential.

What is claimed is:

1. A capillary alginate gel (Capgel) biomaterial-ink for 3D printing, comprised of alginate and optionally a biopolymer(s), wherein the biopolymer is, optionally, gelatin,
wherein the Capgel is in a 3-D form comprising at least one surface that is coated with a polyelectrolyte(s) and/or polyelectrolyte complex(es), wherein the polyelectrolyte is optionally, poly-L-lysine comprising a medium molecular weight (MW=30-70 kDa) positively-charged poly-L-lysine (about 0.01% to about 0.1%, w/v, or, optionally, about 0.05% w/v),
wherein, optionally, the Capgel comprises a plurality of continuous parallel microtubular capillaries having a first end and a second end,
wherein, optionally, the diameter of each of the continuous parallel microtubular capillaries is within the range of about 10 μm to about 300 μm,
wherein, optionally, the diameters of each of the continuous parallel microtubular capillaries may be different,
wherein, optionally, any one diameter of each of the continuous parallel microtubular capillaries varies between the first end and the second end, and/or
wherein, optionally, the cross-section of each of the continuous parallel microtubular capillaries is non-circular.

2. The Capgel biomaterial-ink of claim 1, further comprising a plurality of cells.

3. The Capgel biomaterial-ink of claim 1, wherein a surface of particles of sheared slurries of the Capgel coated with poly-L-lysine interact with a surface of particles of sheared slurries not coated with poly-L-lysine to form a stabilized extruded scaffold.

4. A method of making a Capgel biomaterial-ink for 3D printing, the method comprising steps of:
a. degrading a gelatin solution;
b. adding alginate to the gelatin solution to form an alginate/gelatin solution comprising about 2% alginate and about 2.6 % gelatin;
c. disposing the alginate/gelatin solution in a container coated with dehydrated alginate;
d. covering the container with a porous cover layer soaked with copper (II) sulfate solution;
e. applying copper sulfate solution to the alginate/gelatin solution through the porous cover layer; and
f. allowing the alginate/gelatin solution to set into a capillary alginate gel (Capgel).

5. The method of making the Capgel biomaterial-ink for 3D printing according to claim 4, further comprising steps of
a. cutting the Capgel into segments; and
b. crosslinking the Capgel segments via carbodiimide chemistry.

6. The method of making the Capgel biomaterial-ink for 3D printing according to claim 4 or claim 5 further comprising coating the Capgel segment with a polyelectrolyte(s) and/or polyelectrolyte complex, wherein the polyelectrolyte is optionally poly-L-lysine.

7. The method of claim 6, wherein coating comprises
a. sectioning the Capgel into about 2× about 2× about 2 mm pieces, and putting the pieces in a tube; and/or
b. adding sterile diluted solutions of medium molecular weight (MW=30-70 kDa) positively-charged poly-L-lysine (about 0.01% to about 0.1%, w/v, or, optionally, about 0.05% w/v) to the tube.

8. The method of making the Capgel biomaterial-ink for 3D printing according to any of claims 3-7, further comprising loading segments of Capgel into an extrusion device comprising a reservoir and a needle.

9. The method of making the Capgel biomaterial-ink for 3D printing according to claim 8, further comprising extruding the segments of Capgel onto a substrate.

10. Use of a Capgel biomaterial-ink for 3D printing made according to any of claims 6-9 for producing poly-L-lysine coated Capgel particles as microspheres and microcapsules for encapsulation and delivery of drugs and cells or as injectable biomaterial tissue scaffolds.

11. The method of any of claims 4-9, further comprising loading a plurality of cells into the Capgel biomaterial-ink prior to extruding.

12. The method of 9, further comprising applying cells to the extruded Capgel.

13. A method of 3-D printing, comprising loading a Capgel biomaterial-ink of any of claims 1-3 into an extrusion device comprising a reservoir and, optionally, a needle, and extruding the Capgel biomaterial-ink onto a surface.

14. A method of making a Capgel biomaterial-ink for 3D printing, the method comprising steps of:
(a) adding degraded alginate to a biopolymer solution to form an alginate/biopolymer solution;
(b) disposing the alginate/biopolymer solution in a container;
(c) applying a copper containing solution to the alginate/biopolymer solution; and
(d) subjecting the alginate/biopolymer solution from step (c) under conditions to permit the alginate/biopolymer solution to set into a capillary alginate gel (Capgel).

15. The method of claim 14, wherein the alginate/biopolymer solution comprises a wt % ratio of 0.5-1.0:1.0-2 alginate to biopolymer.

16. The method of claim 14 or 15, wherein the biopolymer is gelatin.

17. The method of claim 16, wherein the alginate/biopolymer solution comprises about 2% alginate and about 2.6 % gelatin.

18. The method of any of claims 14-17, wherein the container in which the alginate/biopolymer solution is disposed is coated with dehydrated alginate.

19. The method of any of claims 14-18, wherein the copper containing solution comprises copper sulfate.

20. The method of any of claims 14-19, further comprising covering the container with a porous cover layer soaked with copper (II) sulfate solution.

21. The method of any of claims 14-20, further comprising steps of
a. cutting the Capgel into segments; and
b. crosslinking the Capgel segments via carbodiimide chemistry a polyelectrolyte(s) and/or polyelectrolyte complex, wherein the polyelectrolyte is optionally poly-L-lysine.

22. The method of claim 21, wherein the polyelectrolyte comprises poly-L-lysine.

23. An extruded shear slurries of a Capgel of any of claims 1-3, or a Capgel made by any of the methods of claims 3-20.

24. A method of 3-D printing, comprising loading a Capgel biomaterial-ink into an extrusion device comprising a reservoir and, optionally, syringe barrel with tip/needle, and extruding the Capgel biomaterial-ink onto a surface, wherein the Capgel biomaterial-ink is comprised of alginate and optionally a biopolymer, wherein the biopolymer is, optionally, gelatin,
wherein, optionally, the Capgel is in a 3-D form comprising at least one surface that is coated with a polyelectrolyte(s) and/or polyelectrolyte complex(es), the polyelectrolyte optionally comprising poly-L-lysine,
wherein, optionally, the Capgel comprises a plurality of continuous parallel microtubular capillaries having a first end and a second end,
wherein, optionally, the diameter of each of the continuous parallel microtubular capillaries is within the range of about 10 μm to about 300 μm,
wherein, optionally, the diameters of each of the continuous parallel microtubular capillaries may be different,
wherein, optionally, any one diameter of each of the continuous parallel microtubular capillaries varies between the first end and the second end, and/or
wherein, optionally, the cross-section of each of the continuous parallel microtubular capillaries is non-circular.

25. A capillary alginate gel (Capgel) biomaterial-ink for 3D printing, comprised of alginate and optionally a biopolymer(s), wherein the biopolymer is, optionally, gelatin,
wherein the Capgel is in a 3-D form comprising at least one surface that is coated with a polyelectrolyte(s) and/or polyelectrolyte complex(es), wherein the polyelectrolyte is optionally, poly-L-lysine comprising a medium molecular weight (MW=30-70 kDa) positively-charged poly-L-lysine (about 0.01% to about 0.1%, w/v, or, optionally, about 0.05% w/v),
wherein, optionally, the Capgel comprises a plurality of continuous parallel microtubular capillaries having a first end and a second end,
wherein, optionally, the diameter of each of the continuous parallel microtubular capillaries is within the range of about 10 μm to about 300 μm,
wherein, optionally, the diameters of each of the continuous parallel microtubular capillaries may be different,
wherein, optionally, any one diameter of each of the continuous parallel microtubular capillaries varies between the first end and the second end, and/or
wherein, optionally, the cross-section of each of the continuous parallel microtubular capillaries is non-circular.
